SEQUENCE LISTING (18 sequences)

SEQ ID NO: 1 - DNA, 20 bp, Homo sapiens
tggtgaaaccccgtctctac

SEQ ID NO: 2 - DNA, 20 bp, Homo sapiens
cgcccggctaatttttgtat

SEQ ID NO: 3 - DNA, 28 bp, Homo sapiens
ttttttttggtgtttgtttttttgatta

SEQ ID NO: 4 - DNA, 25 bp, Homo sapiens
caccaacctcataaccttatcacac

SEQ ID NO: 5 - DNA, 21 bp, Homo sapiens
gtaaggcgttcgagaaagcgt

SEQ ID NO: 6 - DNA, 21 bp, Homo sapiens
cctaaaacaaaaaaccgcgca

SEQ ID NO: 7 - DNA, 23 bp, Homo sapiens
gtttaattttcggtttcgtcgtc

SEQ ID NO: 8 - DNA, 17 bp, Homo sapiens
ctcccgacgtcgctacg

SEQ ID NO: 9 - DNA, 20 bp, Homo sapiens
cgccgaggcggttttttgcg

SEQ ID NO: 10 - DNA, 21 bp, Homo sapiens
cggttttcgttcgttttttcg

SEQ ID NO: 11 - DNA, 20 bp, Homo sapiens
gtaacttccgccttctacgc

SEQ ID NO: 12 - DNA, 20 bp, Homo sapiens
cgccgagggttcgtttatcg

SEQ ID NO: 13 - DNA, 27 bp, Homo sapiens
tttgtttttttgattaggtgtttaaga

SEQ ID NO: 14 - DNA, 21 bp, Homo sapiens
caccaacctcataaccttatc

SEQ ID NO: 15 - DNA, 21 bp, Homo sapiens
ccacggacgatagtgttgtgg

SEQ ID NO: 16 - DNA, 22 bp, Homo sapiens
ctattgttggatcatattcgtc

SEQ ID NO: 17 - DNA, 18 bp, Homo sapiens
ggtagttggagctggtca

SEQ ID NO: 18 - DNA, 20 bp, Homo sapiens
gcgcgtccacgtaggcaaga

(19) United States
(12) Patent Application Publication
(10) Pub. No.: US 2014/0057262 A1
(43) Pub. Date: Feb. 27, 2014

(54) METHODS AND MATERIALS FOR USING THE CONTENTS OF PHAGOCYTES TO DETECT NEOPLASMS

(71) Applicants: David A. Ahlquist, Rochester, MN (US); William R. Taylor, Lake City, MN (US)

(72) Inventors: David A. Ahlquist, Rochester, MN (US); William R. Taylor, Lake City, MN (US)

(73) Assignee: Mayo Foundation for Medical Education and Research, Rochester, NY (US)

(21) Appl. No.: 14/004,952

(22) PCT Filed: Mar. 14, 2012
(86) PCT No.: PCT/US2012/029073

§ 371 Date: Nov. 12, 2013

Related U.S. Application Data
(60) Provisional application No. 61/452,350, filed on Mar. 14, 2011.

(51) Int. Cl. C12Q 1/68

(52) U.S. Cl. CPC C12Q 1/6886
USPC 435/6.11

(57) ABSTRACT

This document provides methods and materials related to detecting premalignant or malignant neoplasms (e.g., colorectal and pancreatic cancer). For example, methods and materials for assessing the contents of phagocytes for the presence of one or more biological markers (e.g., Alu repeats or methylated nucleic acid) of premalignant or malignant neoplasms are provided.

### CROSS-REFERENCE TO RELATED APPLICATIONS

This application claims the benefit of U.S. Provisional Application Ser. No. 61/452,350, filed Mar. 14, 2011. The disclosure of the prior application is considered part of (and is incorporated by reference in) the disclosure of this application.

### BACKGROUND

1. Technical Field

This document relates to methods and materials involved in detecting premalignant or malignant neoplasms (e.g., colorectal and pancreatic cancer). For example, this document relates to methods and materials involved in assessing the contents of phagocytes for the presence of one or more biological markers (e.g., Alu repeats or methylated nucleic acid) of premalignant or malignant neoplasms.

2. Background Information

Cancer is the number one overall killer for those younger than age 85. For essentially all cancers, detection at the earliest stage results in improved cure rates with therapy; and detection and treatment of precursor lesions has the potential to prevent cancer altogether.

### SUMMARY

This document provides methods and materials related to detecting premalignant or malignant neoplasms (e.g., colorectal and pancreatic cancer). For example, this document provides methods and materials for assessing the contents of phagocytes for the presence of one or more biological markers (e.g., Alu repeats or methylated nucleic acid) of premalignant or malignant neoplasms. The detection of such a marker in a sample of phagocytes from a mammal can allow a clinician to diagnose cancer at an early stage. In addition, the analysis of a phagocyte sample obtained from, for example, a standard blood sample can be much less invasive and much more efficient than other types of diagnostic techniques such as endoscopy.

This document is based, in part, on the discovery that the contents of phagocytes can be assessed for the presence of biological markers of cancer or pre-cancerous and that the presence of such markers within phagocytes can be used to discriminate mammals with cancer or pre-cancer from mammals without cancer or pre-cancer. In some cases, a combination of markers found within phagocytes can be used to identify the location and nature of the neoplasm without additional cancer screening techniques such as MRI, endoscopic analysis, and tissue biopsy techniques.

In general, one aspect of this document features a method for assessing a mammal for a premalignant or malignant neoplasm. The method comprises, or consists essentially of, (a) determining whether or not phagocytes of a sample from a mammal contain a phagocytized marker indicative of the presence of the neoplasm, (b) classifying the mammal as having the neoplasm if the phagocytes contain the phagocytized marker, and (c) classifying the mammal as not having the neoplasm if the phagocytes do not contain the phagocytized marker. The mammal can be a human. The neoplasm can originate from a lung or airway, a gastrointestinal tract, a breast, an ovary, a uterus, a prostate, a liver, a urinary tract, a musculoskeletal system, a nervous system, a bone marrow, or a connective tissue. The neoplasm can be colorectal cancer. The phagocytes can be monocytes, macrophages, neutrophils, dendridic cells, or mast cells. The phagocytes can be macrophages. The sample can be a blood sample. The sample can be buffy coat sample. The phagocytized marker can be a nucleic acid marker. The nucleic acid marker can be an increased level of human DNA as assayed by Alu repeats or β-actin. The nucleic acid marker can be an increased level of methylated DNA. The phagocytes can contain the phagocytized marker, and the method can comprise classifying the mammal as having the neoplasm.

In another aspect, this document features a method for identifying a mammal as having a premalignant or malignant neoplasm. The method comprises, or consists essentially of, (a) detecting the presence of a phagocytized marker within phagocytes of a sample from a mammal, wherein the phagocytized marker is indicative of the presence of the neoplasm, and (b) classifying the mammal as having the neoplasm based at least in part on the presence of the phagocytized marker. The mammal can be a human. The neoplasm can originate from a lung or airway, a gastrointestinal tract, a breast, an ovary, a uterus, a prostate, a liver, a urinary tract, a musculoskeletal system, a nervous system, a bone marrow, or a connective tissue. The neoplasm can be colorectal cancer. The phagocytes can be monocytes, macrophages, neutrophils, dendridic cells, or mast cells. The phagocytes can be macrophages. The sample can be a blood sample. The sample can be buffy coat sample. The phagocytized marker can be a nucleic acid marker. The nucleic acid marker can be an increased level of human DNA as assayed by Alu repeats or β-actin. The nucleic acid marker can be an increased level of methylated DNA.

In another aspect, this document features a method for identifying a mammal as having a premalignant or malignant neoplasm. The method comprises, or consists essentially of, (a) lysing phagocytes obtained from a mammal under conditions that lyse few, if any, nuclei of the phagocytes, thereby obtaining a lysed phagocyte sample, (b) detecting the presence of a marker indicative of the presence of the neoplasm within the lysed phagocyte sample, and (c) classifying the mammal as having the neoplasm based at least in part on the presence of the marker within the lysed phagocyte sample. The mammal can be a human. The neoplasm can originate from a lung or airway, a gastrointestinal tract, a breast, an ovary, a uterus, a prostate, a liver, a urinary tract, a musculoskeletal system, a nervous system, a bone marrow, or a connective tissue. The neoplasm can be colorectal cancer. The phagocytes can be monocytes, macrophages, neutrophils, dendridic cells, or mast cells. The phagocytes can be macrophages. The marker can be a nucleic acid marker. The nucleic acid marker can be an increased level of human DNA as assayed by Alu repeats or β-actin. The nucleic acid marker can be an increased level of methylated DNA.

In another aspect, this document features methods for using the techniques described herein in various applications including, without limitation, general population screening and surveillance of high risk subsets of a population.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the description below. Other features, objects, and advantages of the invention will be apparent from the description and from the claims.

### DETAILED DESCRIPTION

This document provides methods and materials related to detecting premalignant or malignant neoplasms (e.g., colorectal and pancreatic cancer). For example, this document provides methods and materials for assessing the contents of phagocytes for the presence of one or more biological markers (e.g., Alu repeats or methylated nucleic acid) of premalignant or malignant neoplasms. Examples of such premalignant or malignant neoplasms that can be detected using the methods and materials provided herein include, without limitation, cancers or pre-cancers of the lung, gastrointestinal tract (e.g., colorectal or pancreatic cancers or pre-cancers), breast, ovary (e.g., ovarian cancer), uterus (e.g., uterine cancer), prostate, liver, urinary tract (e.g., kidney cancer or cancers of the bladder), musculoskeletal system (e.g., sarcomas of muscle or bone), and connective tissue. Any mammal can be assessed using the methods and materials provided herein including, without limitation, humans, monkeys, apes, horses, cows, pigs, sheep, dogs, cats, rats, mice, and rabbits.

As described herein, the presence of one or more markers indicative of a premalignant or malignant neoplasm within the contents of phagocytes can indicate the presence of the premalignant or malignant neoplasm. In some cases, a panel of site-specific neoplasm (e.g., cancer) markers can be used to identify both the presence of a neoplasm in a mammal being tested and the type and/or location of the neoplasm. It will be appreciated that the methods and materials provided herein can be used to detect neoplasm markers from phagocytes of a mammal having a combination of different neoplasms. For example, the methods and materials provided herein can be used to detect nucleic acid and polypeptide markers present within phagocytes of a human having lung and stomach neoplasms.

In some cases, the methods and materials provided herein can be used to quantify multiple markers detected from the contents of phagocytes to yield high sensitivity for detection of lesions (e.g., neoplasms), while preserving high specificity. Such methods can include, for example, multi-plex PCR assays, high-speed deep sequencing, various array technologies, and other approaches; and statistical methods to analyze multi-marker panels can include a logistic model that adjusts specificity cut-offs based on age, gender, or other variables in a target population to be tested or screened.

In some cases, the methods and materials provided herein can be used to determine whether a mammal (e.g., a human) has colorectal cancer or pancreatic cancer based on the profile of markers used. For example, carcino-embryonic antigen, methylated BMP3, or mutant BRAF markers present within the contents of phagocytes might be used to identify a mammal as likely having colorectal cancer; mutant p16 could indicate pancreatic cancer; methylated p16 could signal the presence of esophageal cancer; and methylated CDKN2A could be specific for lung cancer.

Any appropriate method can be used to obtain phagocytes from a mammal (e.g., human) to be tested. Phagocytes can be isolated from various samples including, without limitation, blood, tissue, stool, urine, cerebrospinal fluid, and sputum. For example, a standard blood sample can be collected, and a buffy coat sample can be prepared. In some cases, a crude sample that includes phagocytes as well as other cell types can be used as described herein. For example, sample of peripheral blood mononuclear cells (PBMCs) can be used. In some cases, intact white blood cell preparations that have been viably stored can be used. In some cases, cell isolation techniques can be used to obtain particular cell type such as monocytes or macrophages. For example, cell isolation techniques can be used to obtain monocyte/macrophage subpopulations from crude buffy coat extracts. In some cases, phagocytes can be further purified into particular phagocyte populations (e.g., monocytes, macrophages such as CD16⁺ cells, neutrophils, dendridic cells, or mast cells) or can be used as a mixture of phagocytes.

Any appropriate method can be used to assess the contents of the phagocytes. For example, the phagocytes can be treated with a buffer designed to lyse the plasma membrane of the phagocytes while having little or no ability to lyse the nucleus of the phagocytes. Examples of such buffers include, without limitation, Tris buffers containing nonionic surfactants or detergents such as NP-40, TritinX-100, or Tween-20, Qiagen Qproteome buffers, and plasma membrane lysis buffers (e.g., 50 mM Tris-Cl (pH7.5), 150 mM NaCl, 1.5 mM MgCl₂, and 0.5% Igepal CA-630). Other buffer formulations may contain nuclease or protease inhibitors to slow the degradation of intracellular contents. In some cases, phagocytes can be subjected to mechanical disruption, such as dounce homogenization, which fragments the plasma membrane but leaves nuclear and organelle membranes intact. Once lysed, the contents of the phagocytes can be separated from the nuclei and assessed for the presence of one or more markers.

Any appropriate method can be used to detect a nucleic acid marker. For example, such methods can involve isolating DNA from a cytosolic sample obtained from lysed phagocytes, separating out one or more particular DNAs from the total DNA, subjecting the DNAs to bisulfite treatment, and determining whether the separated DNAs are abnormally methylated (e.g., hypermethylated or hypomethylated). In some cases, such methods can involve isolating DNA from a cytosolic sample obtained from lysed phagocytes and determining the level of Alu repeats present. In some cases, such methods can involve isolating DNA from a cytosolic sample obtained from lysed phagocytes, and subjecting the DNAs to allele-specific detection assays to determine the presence or absence of a sequence alteration or mutation. It is noted that a single cytosolic sample obtained from lysed phagocytes can be analyzed for one nucleic acid marker or for multiple nucleic acid markers. For example, a cytosolic sample obtained from lysed phagocytes can be analyzed using assays that detect a panel of different nucleic acid markers. In addition, multiple cytosolic samples obtained from lysed phagocytes can be collected from a single mammal and analyzed as described herein.

In some cases, Solid Phase Reversible Immobilization (SPRI) bead technology can be used to enrich samples for particular nucleic acid fragment sizes. In some cases, enriching for short nucleic acid fragments (e.g., less than 300 bp, less than 250 bp, or less than 200 bp) can improve tumor marker detection. In some cases, size selection for fragments (e.g., less than 300 bp, less than 250 bp, or less than 200 bp) can used without performing a monocyte-specific cytoplasmic/nuclear content separation step. For example, size selection for fragments (e.g., less than 300 bp, less than 250 bp, or less than 200 bp) can used to obtain nucleic acid from commonly collected PBMCs without performing a cytoplasmic/nuclear content separation step.

One or more specific nucleic acid fragments can be purified from a nucleic acid preparation using, for example, a modified sequence-specific hybrid capture technique (see, e.g., Ahlquist et al. (2000) Gastroenterol. 119:1219-1227). Such a protocol can involve: (1) adding 300 μL of sample preparation to an equal volume of a 6 M guanidine isothiocyanate solution containing 20 μmol biotinylated oligonucleotides (obtained from, for example, Midland Certified Reagent Co., Midland, Tex.) with sequences specific for the DNA fragments to be analyzed; (2) incubating for two hours at 25° C.; (3) adding streptavidin coated magnetic beads to the solution and incubating for an additional hour at room temperature; (4) washing the bead/hybrid capture complexes four times with 1×B+W buffer (1 M NaCl, 0.01 M Tris-HCl pH 7.2, 0.001 M EDTA, 0.1% Tween 20); and (5) eluting the sequence specific captured DNA into 35 μL-TE (1 mM Tris pH 7.4, 0.1 M EDTA) by heat denaturation of the bead/hybrid capture complexes. In some cases, DNA fragments can be separated based on size, where smaller fragments can represent ingested material undergoing hydrolysis and larger fragments can represent the endogenous leukocyte DNA. Such a protocol can involve separating the isolated DNA on an agarose gel and cutting out bands of specific length. In some cases, SPRI bead technology can be used to obtain DNA fragments of particular sizes. Any other suitable technique also can be used to isolate specific nucleic acid fragments.

Any appropriate method can be used to determine whether a particular nucleic acid marker is present within a sample. For example, QuARTS amplification techniques or SYBR Green qMSP and allele-specific PCR techniques can be used to detect the presence or absence of methylated DNA (e.g., methylated BMP3, NDRG4, or TFPI2 DNA), DNA mutations (e.g., KRAS2 or BRAF DNA mutations), or microRNA levels within nucleic acid samples.

Any appropriate method can be used to determine whether a particular DNA is hypermethylated or hypomethylated. Standard PCR techniques, for example, can be used to determine which residues are methylated, since unmethylated cytosines converted to uracil are replaced by thymidine residues during PCR. PCR reactions can contain, for example, 10 μL of captured DNA that either has or has not been treated with sodium bisulfite, 1×PCR buffer, 0.2 mM dNTPs, 0.5 μM sequence specific primers (e.g., primers flanking a CpG island within the captured DNA), and 5 units DNA polymerase (e.g., Amplitaq DNA polymerase from PE Applied Biosystems, Norwalk, Conn.) in a total volume of 50 μL. A typical PCR protocol can include, for example, an initial denaturation step at 94° C. for 5 minutes, 40 amplification cycles consisting of 1 minute at 94° C., 1 minute at 60° C., and 1 minute at 72° C., and a final extension step at 72° C. for 5 minutes.

To analyze which residues within a captured DNA are methylated, the sequences of PCR products corresponding to samples treated with and without sodium bisulfite can be compared.

The sequence from the untreated DNA will reveal the positions of all cytosine residues within the PCR product. Cytosines that were methylated will be converted to thymidine residues in the sequence of the bisulfite-treated DNA, while residues that were not methylated will be unaffected by bisulfite treatment.

In some cases, a cytosolic sample obtained from lysed phagocytes can be assessed for the presence or absence of a polypeptide marker. For example, any appropriate method can be used to assess a cytosolic sample obtained from lysed phagocytes for a polypeptide marker indicative of a neoplasm. For example, a cytosolic sample obtained from lysed phagocytes can be used in assays designed to detect one or more polypeptide markers. Appropriate methods such as those described elsewhere (Aebersold and Mann, Nature, 422:198-207 (2003) and McDonald and Yates, Dis. Markers, 18:99-105 (2002)) can be adapted or designed to detect polypeptides in a cytosolic sample obtained from lysed phagocytes. For example, single-reaction monitoring using a TSQ mass spectrometer can specifically target polypeptides in a cytosolic sample obtained from lysed phagocytes. High Resolution instruments like the LTQ-FT or LTQ orbitrap can be used to detect polypeptides present in a cytosolic sample obtained from lysed phagocytes.

In some cases, a ratio of particular polypeptide markers can be determined and used to identify a mammal having an aerodigestive cancer (e.g., a colorectal cancer or a pancreatic cancer). In some cases, a matrix marker panel can be used to identify mammals having an aerodigestive cancer (e.g., a colorectal cancer or a pancreatic cancer). In some cases, such panel also can identify the location of the aerodigestive cancer. Such a panel can include nucleic acid markers, polypeptide markers, and combinations thereof and can provide information about a mutated marker gene, the mutated region of the marker gene, and/or type of mutation.

In some cases, the methods and materials provided herein can be used in combination with an assessment of free circulating nucleic acid and/or an assessment of circulating cells for tumor cells to discriminate mammals with cancer or pre-cancer from mammals without cancer or pre-cancer. In some cases, such a combination of techniques can be used to identify the location and nature of the neoplasm without additional cancer screening techniques such as MRI, endoscopic analysis, and tissue biopsy techniques.

The invention will be further described in the following examples, which do not limit the scope of the invention described in the claims.

### EXAMPLES

### Example 1

### Detecting Colorectal Cancer by Evaluating Leukocytes Obtained from Blood

Circulating leukocytes from patients with or without colorectal cancer were evaluated to determine if circulating leukocytes contain detectable levels of nucleic acid cancer markers that are capable of discriminating patients with colorectal cancer from healthy control patients. By selectively lysing the plasma membrane of circulating leukocytes, thereby leaving the nucleus intact, ingested human DNA and a tumor marker (e.g., methylated TFPI2 gene) were successfully isolated from the cytosol of buffy-coat leukocytes. Such ingested DNA analytes were capable of discriminating patients with colorectal cancer from healthy control patients.

### Methods and Materials

Crude buffy coat (e.g., white blood cell) samples from 21 patients presenting with stage 3-4 colorectal cancers were analyzed along with 11 buffy coat samples from colonoscopically normal individuals with similar demographics. Contaminating red blood cells and platelets were removed from 500 μL of buffy coat by room temperature centrifugation at 500×g for 10 minutes and washing pelleted cells several times with equal volumes of 1×PBS containing 10 mM EDTA and 0.1% BSA. During the final wash, an aliquot was removed from each of the samples, and the number of cells was counted. Once the RBCs and platelets were removed, the remaining leukocytes were incubated with 1 mL of Qiagen Qproteome extraction buffer CE1 (minus protease inhibitor) for 10 minutes at room temperature with mild mixing. This buffer allowed for the selective disruption of the plasma membrane and release of cytosolic contents. The mixture was centrifuged at 1000×g for 10 minutes, and the supernatant removed and stored frozen for subsequent analyses. The pellet was resuspended in 1 mL of CE2 buffer, which completely lyses the plasma membrane as well as all organelle membranes except for the nuclear membrane. Samples were incubated for 30 minutes at room temperature with mixing, and then pelleted at 6000×g for 10 minutes. Supernatants were stored as before. The nuclear pellet was resuspended in 500 μL of CE3 buffer, which solubilizes the nuclear membrane and releases nuclear DNA. Samples were incubated at room temperature for 10 minutes with mixing and centrifuged at 6800×g for 10 minutes. The liquid fraction was stored, and the remaining pellet discarded.

DNA was purified from each of these fractions (CE1, CE2, and CE3 fractions) using the QIAamp circulating nucleic kit. The columns and buffers in this procedure were optimized for isolation of low concentrations of short DNA (>20 base pairs), which can be ideal for potentially ingested and degraded cytosolic DNA. Proteinase digestion of contaminating proteins was followed by column binding and washing steps performed on a vacuum apparatus, and subsequent elution in 50 μL of tris buffer.

The genomic human DNA content from each of the fractions was measured by a quantitative analysis of a common short interspersed element (SINE) in the human genome, the Alu repeat. Primers amplifying a unique 45 base pair Alu segment were designed to have the following sequences:

(SEQ ID NO: 1)
Forward: 5′-TGGTGAAACCCCGTCTCTAC-3′
(SEQ ID NO: 2)
Reverse: 5′-CGCCCGGCTAATTTTTGTAT-3′

Amplification reactions were performed using a Roche FastStart Taq DNA polymerase in a SYBR Green 1 master mix format specifically formulated for the LightCycler 480 (Roche). PCR was performed in 25 μL reaction volumes, with 2 μL of sample template, under the following conditions: 95° C. for 3 minutes followed by 50 cycles of 95° C. for 30 seconds, 60° C. for 30 seconds, and 72° C. for 30 seconds. Serial dilutions of an hgDNA control (Novagen) were used to construct quantitative standards. Threshold cycle numbers were determined using LightCycler software. The quantification for each sample fraction was used to determine the genomic equivalents by comparison to the standards.

The remaining sample fractions (˜45 μL/each) were concentrated by speedvac and bisulfite converted using the Epitect procedure (Qiagen), with some buffer modifications specific to treating and recovering degraded DNA as recommended by the manufacturer. Desulfonated DNA was eluted into 20 μL of EB buffer and frozen.

The bisulfite treated DNA was divided into two fractions, one which was tested for β-actin (ACTB) copy number by qPCR, and the other for methylated TFPI2, a marker which appears at high frequency in colorectal tumors. The primers were designed as follows:

ACTB (~100 bp)
(SEQ ID NO: 3)
Forward: 5′-TTTTTTTTGGTGTTTGTTTTTTTGATTA-3′
(SEQ ID NO: 4)
Reverse: 5′-CACCAACCTCATAACCTTATCACAC-3′
TFPI2 (~60 bp)
(SEQ ID NO: 5)
Forward: 5′-GTAAGGCGTTCGAGAAAGCGT-3′
(SEQ ID NO: 6)
Reverse: 5′-CCTAAAACAAAAAACCGCGCA-3′

The ACTB amplification is methylation insensitive and gives an indication of the post-bisulfite copy number of each of the samples, which can be used to normalize the methylation specific data.

Eight μL of each fraction was run in single-plex format for both amplifications. Both utilized FastStart polymerase with SYBR Green 1 in 30 μL reactions, with 0.5 μM primer concentrations. ACTB conditions were 95° C. for 3 minutes followed by 50 cycles of 95° C. for 30 seconds, 60° C. for 30 seconds, and 72° C. for 30 seconds. TFPI2 conditions were 95° C. for 3 minutes followed by 70 cycles of 95° C. for 30 seconds, 65° C. for 30 seconds, and 72° C. for 30 seconds. For both amplifications, 5-fold serial diluted bisulfite-treated methylated control DNA (Millipore) samples were used as quantification standards. Bisulfite-treated unmethylated DNA was used as a negative control for the TFPI2 assay.

Data were sorted into common fractions based on extraction buffers (CE1, CE2, and CE3) and analyzed in both absolute and normalized formats.

### Results

The Alu derived genomic equivalents from the CE1 fraction resulted in the most discriminating data. Determining the levels of Alu repeats, which number around 1 million per haploid genome, represents a way of measuring, at very high analytical sensitivity, total DNA content in a sample fraction. Genomic equivalents can be derived by dividing total repeat copies by 1×10⁶. The 11 samples from normal controls resulted in a median value of 94 copies/μL, while the 21 samples from cancer patients resulted in a median of 2110 copies/μL. At an 82% cutoff, 76% of all cancer patients were detected. The CE2 fraction had an equivalent sensitivity. The nuclear fraction, however, exhibited no discrimination. For TFPI2, absolute CE2 copies detected 52% of the cancer patients at the 82% cutoff, and 48% of the cancer patients at a 91% cutoff. Nuclear TFPI2 appeared not to discriminate normal controls from cancer patients. When the data was normalized to total DNA present (ACTB), TFPI2 sensitivities in both CE1 and CE2 fractions markedly decreased, indicating absolute copy number may be a more reliable indicator.

TABLE 1
Sensitivities at 82% specificity.

| Cytosolic | ALU 45* | mTFPI2 | |
|---|---|---|---|
| Fraction | (absolute) | (absolute) | mTFPI2/ACTB |
| CE1 (cytosol 1) | 76% | 19% | 29% |
| CE2 (cytosol 2) | 76% | 52% | 29% |
| CE3 (nuclei) | 0% | 10% | 43% |

*ALU 45 refers to 45 base-pair length ALU amplicons (also called “short human DNA”)

These results demonstrate that non-nuclear DNA and methylated DNA tumor markers can be recovered in circulating leukocytes. These results also demonstrate that cytosolic DNA markers can discriminate patients with colorectal cancer from healthy control patients.

### Example 2

### Increasing the Amount of Analyte Available for Assay

With bi-allelic methylation markers, the absolute copy numbers after background subtraction were very low (<20 copies/250 μL crude buffy coat). One or more of the following are performed to increase the amount of analyte available for assay: larger volumes of starting material, different buffer formulations which allow for less sample degradation, addition of carrier molecules to increase purification recoveries, reduction of non-specific noise in the MSP step using, for example, qInvader probes, and/or nested amplification. In some cases, a whole genome amplification step can increase analyte levels by 100- to 1000-fold. In some cases, a further lysis step is performed during the preparation steps to recover more analytes contained in phagosomes. Isolating the leukocyte subset, e.g. phagocytes, is also a measure that can be used to enrich capture of target analytes (see Example 3).

### Example 3

### Isolating Phagocytes from the Larger Leukocyte Population

Phagocytic subpopulations include monocytes, macrophages, neutrophils, dendridic cells, and mast cells. One mL of blood contains about 6 million phagocytes. In Example 1, DNA was extracted from unfractionated leukocytes, which include non-phagocytic white blood cells such as lymphocytes. Cytosolic DNA from these non-phagocytic cells may contribute to background noise in the MSP reaction. In some embodiments, an assay is performed using phagocyte cell populations that are selectively isolated by, for example, density gradient centrifugation, adherence to solid supports, immuno-capture, and/or flow cytometry. Solid tumors are composed of up to 50% leukocytes; the most represented fractions being lymphocytes and macrophages. Antibodies such as anti-CD14 and anti-CD16 antibodies are used to immune-capture macrophages, which may predominantly include tumor-specific tissue resident macrophages.

### Example 4

### Detecting Patients with Primary Sporadic Colorectal Cancer

### Sample Collection

Six samples were selected from Mayo's Familial Cancer Program (FCP) biobank. The samples came from patients with primary sporadic colorectal cancer. These samples were samples from which PBMCs were isolated by density centrifugation (Lymphoprep, MP Biomedicals LLC) and viably frozen. In addition, six apheresis trima cone samples were acquired from Transfusion Medicine. These PBMC samples originated from disease-free individuals participating in blood donation, where leukocyte reduction occurred as part of the collection process. Monocytes were isolated from PBMCs by incubation with anti-CD14 immunomagnetic beads (Miltenyi Biotec) per the manufacturer's protocol and were selected using the AutoMACS Separator (MiltenyiBiotec). CD14⁺ yields were 8-40% of total PBMCs, and absolute cell counts were 200,000 to 4,000,000. One FCP sample did not yield any monocytes, and it was determined that the sample had been compromised either during preparation or storage.

### Cytoplasmic DNA Isolation and Bisulfite Treatment

CD14⁺ cells were treated for 5 minutes with a plasma membrane lysis buffer (50 mM Tris-Cl, pH7.5, 150 mM NaCl, 1.5 mM MgCl₂, and 0.5% Igepal CA-630). The nuclei were pelleted, and the cytoplasmic DNA fraction separated and subsequently purified using the QIAamp Circulating Nucleic Acid kit (Qiagen, Inc.). The entire elution volume was then treated with sodium bisulfite for 5 hours, column purified, desulfonated, and eluted in EB buffer (Qiagen—Epitect).

### QuARTS Assay

All samples were further diluted 1:10 in EB buffer, and 10 μL aliquots were amplified using Exact Science's QuARTS (Quantitative Allele-specific Real-time Target and Signal) method. In addition, methylation positive and negative controls were run at levels approximating 10,000 copies each. Three primer/probe systems were used (Table 2): two for the colorectal cancer-specific methylation markers BMP3 and NDRG4, and one for a control β-actin sequence not affected by methylation.

TABLE 2
Oligonucleotide sequences used in QuARTS assays.

| Assay | Oligo name | Sequence |
|---|---|---|
| Methylated BMP3 | Forward primer | 5′-GTT TAA TTT TCG GTT TCG TCG TC-3′ |
| | | (SEQ ID NO: 7) |
| | Reverse primer | 5′-CTC CCG ACG TCG CTA CG-3′ |
| | | (SEQ ID NO: 8) |
| | Probe with 3′ C6 | 5′-CGC CGA GGC GGT TTT TTG CG-3′ |
| | | (SEQ ID NO: 9) |
| Methylated NDRG4 | Forward primer | 5′-CGG TTT TCG TTC GTT TTT TCG-3′ |
| | | (SEQ ID NO: 10) |
| | Reverse primer | 5′-GTA ACT TCC GCC TTC TAC GC-3′ |
| | | (SEQ ID NO: 11) |
| | Probe with 3′ C6 | 5′-CGC CGA GGG TTC GTT TAT CG-3′ |
| | | (SEQ ID NO: 12) |
| Bisulfite-treated ACTB | Forward primer | 5′-TTT GTT TTT TTG ATT AGG TGT TTA AGA-3′ |
| | | (SEQ ID NO: 13) |
| | Reverse primer | 5′-CAC CAA CCT CAT AAC CTT ATC-3′ |
| | | (SEQ ID NO: 14) |
| | Probe with 3′ C6 | 5′-CCA CGG ACG ATA GTG TTG TGG-3′ |
| | | (SEQ ID NO: 15) |

Single-plex QuARTS reactions were run on the Roche 480 LightCycler (Roche, Inc.) using 500 nmol/L of each primer and detection probe, 300 nmol/L FAM and Red FRET cassettes (Hologic), 6.675 ng/μL Cleavase® (Hologic), 1 U GoTaq® DNA polymerase (Promega), 10 mmol/L 3-(n-morpholino) propanesulfonic acid (MOPS), 7.5 mmol/L MgCl₂, and 250 μmol/L of each dNTP. QuARTS cycling conditions were 95° C. for 3 minutes followed by 10 cycles of 95° C. for 20 seconds, 67° C. for 30 seconds, and 70° C. for 30 seconds. After completion of the 10 cycles, an additional 37 cycles at 95° C. for 20 seconds, 53° C. for 1 minute, and 70° C. for 30 seconds were performed.

### Data Analysis

Strand numbers of each gene were determined by comparing to a standard curve using known copies of plasmid DNA. Strand numbers of the methylation-specific markers were normalized to input DNA by dividing by β-actin strands.

### Results

Methylated NDRG4 normalized strands cleanly discriminated all the FCP cancers from the apheresis normal (Table 3). There were no false positive or false negative results for these samples. Thus, the sensitivity and specificity were both 100%. Methylated BMP3 was not present in the samples, which was not unusual as it was used as a complimentary marker to NDRG4 for CRC. BMP3 amplified as expected in the M+ control (Table 3).

TABLE 3
CD14⁺ Cytoplasmic Markers (FCP vs. Trima cone).

| | | | |
|---|---|---|---|
| Methylated | Methylated | ACTB | NDRG4/ |
| BMP3 (copies) | NDRG4 (copies) | (copies) | ACTB* |

CD14⁺ Apheresis Samples

| | | | | |
|---|---|---|---|---|
| 1 | 0 | 86.5 | 40400 | 21 |
| 2 | 0 | 149.1 | 27350 | 55 |
| 3 | 0 | 109.2 | 63200 | 17 |
| 4 | 0 | 86.2 | 35260 | 24 |
| 5 | 0 | 94.4 | 28920 | 33 |

| | |
|---|---|
| Average: | 30 |
| Standard Deviation: | 15 |

FCP Samples

| | | | | |
|---|---|---|---|---|
| 1 | 0 | 14.63 | 971 | 151 |
| 2 | 0 | 8.38 | 577 | 145 |
| 3 | 0 | 11.55 | 1266 | 91 |
| 4 | 0 | 21.7 | 861 | 252 |
| 5 | 0 | 12.93 | 1044 | 124 |

| | |
|---|---|
| Average: | 153 |
| Standard Deviation: | 60 |

Controls

| | | | |
|---|---|---|---|
| M+ (~10K | 24800 | 16900 | 5630 |
| copies) | | | |
| M− (~10K | 0.01 | 10.9 | 10200 |
| copies) | | | |
| no template | 0 | 0 | 0 |

*NDRG4/ACTB represents the methylated NDRG4 copies divided by the ACTB copies times 10,000

The 1:10 dilutions were found to amplify more efficiently than the samples run straight, possibly due to carryover inhibition from the bisulfite reaction. An additional purification using SPRI beads (Becton-Dickenson, Inc.) mitigated this effect and allowed undiluted samples to be loaded in the QuARTS reaction.

The results demonstrate that cytosolic DNA preparations (e.g., from CD14⁺ monocytes) from a cohort of patients with sporadic colon cancers contain higher percentages of the NDRG4 methylation marker than monocytes harvested from normal individuals. The numbers observed validate the methods used to isolate the marker DNA and the overall disclosure that phagocytes can be carriers of tumor derived material. These results also demonstrate that samples containing suboptimal cell numbers as well as older samples can be used to identify cancer patients using the methods and materials provided herein since the cells of the FCP samples were isolated and stored many years ago and four of those FCP samples had suboptimal cell counts.

### Example 5

### Circulating Monocytes are Capable of Engulfing Colorectal Cancer Cells

The following was performed to determine whether the subset of circulating monocytes known to convert to macrophages (CD14⁺ monocytes) are capable of engulfing colorectal cancer cells when briefly incubated with cultured tumor cells.

### Sample Collection

Two stocked ATCC CRC cell lines (HCT116 and DLD1) containing a heterozygous mutation in the KRAS2 proto-oncogene (G13D) were thawed and cultured in complete RPMI+10% FCS. A single leukocyte reduction cone sample was acquired from Transfusion Medicine. PBMCs were purified by density centrifugation (Lymphoprep, MP Biomedicals LLC). CD14⁺ monocytes were isolated by incubating PBMCs with anti-CD14 immunomagnetic beads (Miltenyi Biotec) according to the manufacturer's protocol and were selected using the AutoMACS Separator (MiltenyiBiotec). The donor CD14⁺ fraction was cultured as stated above. All cells were grown and subcultured until requisite numbers were obtained.

At this point, separate co-cultures were started by plating 4×10⁷ CD14⁺ monocytes with 2×10⁶ cells from each of the ATCC lines. Fresh CD14⁺ monocytes and cancer cell line control subcultures were also started. Cells were allowed to grow with media supplementation as necessary. After 4 days, cultures were harvested. For the co-cultures, TrypLE (Invitrogen, Inc.) was used to collect the tumor cells along with the free floating cells. Monocytes were re-isolated from the co-cultures. Flow analysis was done on the combined cells of both floating and TrypLE treated attached cells. Free floating cells from the CD14/RPMI/FCS cultures were also flow sorted. Antibody staining on the cells was CD45PE/HLA-DR PerCP/CD14APC.

### DNA Isolation

CD14⁺ monocytes from the co-cultures were treated for 5 minutes with a plasma membrane lysis buffer (50 mM Tris-Cl, pH7.5, 150 mM NaCl, 1.5 mM MgCl₂, and 0.5% Igepal CA-630). The nuclei were pelleted, and the cytoplasmic DNA fraction separated and subsequently purified. Total cellular DNA was isolated from the HCT116 and DLD1 cancer cell positive control cultures and the CD 14⁺ negative control cultures. Free DNA also was isolated from 200 μL aliquots of final media from each of the culture flasks. All DNA purifications were done using the QIAamp Blood Mini kit (Qiagen, Inc.). Samples were eluted in 200 μL of EB buffer. The entire volume was then PEG8000 bead purified at two levels of size selection using the AmPure XP product (Becton-Dickinson, Inc.): 0.5× (>400 bp) and 2× (80-400 bp). Samples were eluted from the beads in 500 μL of EB buffer, and DNA concentrations were determined by Nanodrop absorbance (Thermo Fisher Scientific, Inc.).

### QuARTS Assay

10 μL aliquots of each sample were amplified using Exact Science's QuARTS (Quantitative Allele-specific Real-time Target and Signal) method with reagents specific for the KRAS G13D mutation (Table 4).

TABLE 4
Oligonucleotide sequences used in QuARTS assay.

| Oligo name | Sequence |
|---|---|
| KRAS RP5 | 5′-CTATTGTTGGATCATATTCGTC-3′ |
| | (SEQ ID NO: 16) |
| KRAS 38A P2C | 5′-GGTAGTTGGAGCTGGTCA-3′ |
| | (SEQ ID NO: 17) |
| KRAS 38A A7 | 5′-GCGCGTCCACGTAGGCAAGA-3′ |
| Pb (with 3′ C6) | (SEQ ID NO: 18) |

Single-plex QuARTS reactions were run on the Roche 480 LightCycler (Roche, Inc.) using 500 nmol/L of each primer and detection probe, 300 nmol/L FAM FRET cassettes (Hologic), 6.675 ng/μL Cleavase° (Hologic), 1 U GoTaq® DNA polymerase (Promega), 10 mmol/L 3-(n-morpholino) propanesulfonic acid (MOPS), 7.5 mmol/L MgCl₂, and 250 μmol/L of each dNTP. QuARTS cycling conditions were 95° C. for 3 minutes followed by 10 cycles of 95° C. for 20 seconds, 67° C. for 30 seconds, and 70° C. for 30 seconds. After completion of the 10 cycles, an additional 37 cycles at 95° C. for 20 seconds, 53° C. for 1 minute, and 70° C. for 30 seconds were performed.

### Data Analysis

Strand numbers for the samples were determined by comparing raw counts to those from mutant KRAS plasmid standards at known copy numbers. Strand numbers were normalized by dividing by the amount of DNA used in the reactions. Uptake numbers were derived by comparing normalized co-culture levels to the normalized signal off the pure CRC cultures.

### Results

The cytoplasmic DNA from both sets of co-cultured monocytes was positive for KRAS G13D (Table 5). However, the fraction of KRAS mutant sequences was higher in the short DNA fragment group (<400 base pairs) than in the long fragment group (>400 base pairs). 12.03% and 13.01% of mutant KRAS in short fragment group from monocytes co-cultured with DLD1 and HCT116 cell lines, respectively; and 3.63% and 3.78%, respectively, in long fragment group. The enriched mutant marker in short fragment group demonstrates that DNA is consistent with monocyte (phagosome) metabolism and against contamination by cultured colon cancer cell lines. The monocyte controls were completely negative for the KRAS mutation.

TABLE 5
Circulating monocytes engulf colorectal cancer cells.

| | | | | kras+ | % |
|---|---|---|---|---|---|
| | DNA | ng | kras+ | copies/ | marker |
| sample | size | DNA | copies | ng | uptake |
| monocyte neg control | >400 bp | 56 | 0 | 0 | |
| dld1 pos control | >400 bp | 100 | 12000 | 120 | |
| hct116 pos control | >400 bp | 100 | 12500 | 125 | |
| dld1 monocyte co-culture | >400 bp | 100 | 434 | 4.34 | 3.63 |
| hct116 monocyte co-culture | >400 bp | 100 | 473 | 4.73 | 3.78 |
| monocyte neg control | <400 bp | 18.7 | 0 | 0 | |
| dld1 pos control | <400 bp | 100 | 12000 | 120 | |
| hct116 pos control | <400 bp | 100 | 9840 | 98.4 | |
| dld1 monocyte co-culture | <400 bp | 100 | 1444 | 14.44 | 12.03 |
| hct116 monocyte co-culture | <400 bp | 100 | 1280 | 12.8 | 13.01 |

| Total % uptake | |
|---|---|
| dld1 monocyte co-culture | 15.66 |
| hct116 monocyte co-culture | 16.79 |

The magnetic selection of the CD14⁺ cells from the co-cultures rendered them greater than 98 percent pure. Given that the total percent uptake amounts for DLD1 and HCT116 mutant KRAS were 15.66 and 16.79, respectively, it was concluded that the CD14⁺ monocytes were actively scavenging and internalizing cancer cells and cellular debris.

Taken together, these results demonstrate that tumor marker uptake by phagocytic cells can be replicated in a model system in vitro. They also provide further confirmation of the positive studies with patient derived phagocytes.

### Example 5

### Analysis of Size Selected Nucleic Acid from Crude Buffy Coat Samples

The following was performed to determine if it was possible to detect phagocytized nucleic acid without having to isolate cytosolic or cytoplasmic DNA from purified phagocytes. Total DNA was extracted from 74 crude buffy coat samples from 20 CRC patients, 18 patients with polyps greater than 1 cm in size, and 36 colonoscopy normal individuals. All samples were age/gender matched. 200 μL of sample were used, and the DNA was purified using QIAamp mini kits (Qiagen, Inc.). Ampure XP beads (Becton-Dickinson, Inc.) were used at 0.8× and 2× to size select DNA into two groups: (1) fragments greater than 250 bp; and (2) fragments less than 250 bp. These were then treated with sodium bisulfite for 5 hours and isolated using Epitect columns (Qiagen, Inc.). 10 μL of each sample was amplified with BMP3, NDRG4, and ACTB qInvader primers (Exact Sciences, Inc.) and standardized against plasmid stocks with known copy numbers.

The CRC buffy coat BMP3 DNA had a mean copy number value of 12.2. The polyp BMP3 DNA had a mean copy number value of 8.54; and the normal BMP3 DNA had a mean copy number value of 2.02. For NDRG4, the numbers were 162.38 copies for CRC, 89.34 copies for the polyp samples, and 124.06 copies for the normal samples. The 250 bp size cut-off was not significant for this analysis. These fragment size results may be due to background methylation levels of the markers in the WBC DNA.

During the analysis, data from a methyl deep-sequencing study where buffy coat DNA was included with the marker set including BMP3 and NDRG4 were reassessed. BMP3 exhibited moderate levels of methylation (5-16%) in buffy coats, while NDRG4 was higher (23-34%). It is possible that this level of background methylation contributes to the observed copy numbers in the WBC samples.

Taken together, these results demonstrate that total DNA derived from buffy coat (white blood cell) samples had measureable background methylation for the markers in question, potentially limiting the use of these particular markers when using unpurified patient material. These results also demonstrate that purified cytoplasmic nucleic acid from phagocytes is an effective source of material for performing the tests described herein.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-23. (canceled)

24. A method for identifying a mammal as having a premalignant or malignant neoplasm, wherein said method comprises:
(a) lysing phagocytes obtained from a mammal under conditions that lyse few, if any, nuclei of said phagocytes, thereby obtaining a lysed phagocyte sample,
(b) detecting the presence of a marker indicative of the presence of said neoplasm within said lysed phagocyte sample, and
(c) classifying said mammal as having said neoplasm based at least in part on said presence of said marker within said lysed phagocyte sample.

25. The method of claim 24, wherein said mammal is a human.

26. The method of claim 24, wherein said neoplasm originates from a lung or airway, a gastrointestinal tract, a breast, an ovary, a uterus, a prostate, a liver, a urinary tract, a musculoskeletal system, a nervous system, a bone marrow, or a connective tissue.

27. The method of claim 24, wherein said neoplasm is colorectal cancer.

28. The method of claim 24, wherein said phagocytes are monocytes, macrophages, neutrophils, dendridic cells, or mast cells.

29. The method of claim 24, wherein said phagocytes are macrophages.

30. The method of claim 24, wherein said marker is a nucleic acid marker.

31. The method of claim 30, wherein said nucleic acid marker is an increased level of human DNA as assayed by Alu repeats or β-actin.

32. The method of claim 30, wherein said nucleic acid marker is an increased level of methylated DNA.
